(19) United States
(12) Patent Application Publication
(10) Pub. No.: US 2014/0296990 A1
(43) Pub. Date: Oct. 2, 2014

(54) KNEE ARTHROPLASTY TIBIA COMPONENT AND KNEE ARTHROPLASTY

(71) Applicants: Dein Shaw, Hsinchu (TW); Tze-Hong Wong, Hsinchu (TW); Yi-Cheng Lin, Hsinchu (TW); Tsung-Meng Chen, Hsinchu (TW)

(72) Inventors: Dein Shaw, Hsinchu (TW); Tze-Hong Wong, Hsinchu (TW); Yi-Cheng Lin, Hsinchu (TW); Tsung-Meng Chen, Hsinchu (TW)

(21) Appl. No.: 14/098,402

(22) Filed: Dec. 5, 2013

(30) Foreign Application Priority Data
Mar. 29, 2013 (TW) 102111341

(51) Int. Cl. A61F 2/30; A61F 2/38

(52) U.S. Cl. CPC A61F 2/30749; A61F 2/389
USPC 623/20.21; 623/20.32

(57) ABSTRACT

A tibia component of a knee arthroplasty to be connected to a tibia includes a first tibia connecting structure and a second tibia connecting structure. The first tibia connecting structure can be inserted into a radial groove on the tibia along a radial direction of the tibia to limit the motion of the tibia component in the axial direction of the tibia. The second tibia connecting structure can be inserted into an axial groove on the tibia along the axial direction of the tibia to limit the motion of the tibia component in the radial direction of the tibia. The first and the second connecting structures provide the radial and axial bonding force to the tibia and the knee arthroplasty, so that the amount of bone cement used can be reduced and may beneficial to revision knee arthroplasty operation.

### PRIORITY CLAIM

This application claims the benefit of the filing date of Taiwan Patent Application No. 102111341, filed Mar. 29, 2013, entitled “KNEE ARTHROPLASTY TIBIA COMPONENT AND KNEE ARTHROPLASTY,” and the contents of which is hereby incorporated by reference in its entirety.

### FIELD OF THE INVENTION

The present invention relates to a tibia component and an knee arthroplasty, more particularly, a tibia component which can reduce the use of the bone cement and provide enough bonding force, and an knee arthroplasty that utilizes the tibia component.

### BACKGROUND OF THE INVENTION

Arthritis is the most common chronic disease in the world. The degradation of articular cartilage or inflammation in the connective tissue leads to joint pain and interferes with normal movements of the joint, which is known as arthritis. There are many causes of arthritis, and the problems caused by arthritis are also different. There are about 355 million arthritis patients in the world, with more than 100 million arthritis patients in China. In the U.S., one in five people have arthritis; while in Asia, one in six.

Common of arthritis are (1) rheumatoid arthritis: an autoimmune disease, and (2) osteoarthritis: degenerative arthritis. The most common symptoms of arthritis are joint pain, joint deformity, movement disorder, difficulties in walking, stiffness and swelling. Osteoarthritis usually occurs in the elderly over 50 years old, and with the advent of aging population, osteoarthritis is becoming a more and more serious medical problem. In addition to the elderly, osteoarthritis can occur in labor workers or athletes because of excessive use of their joints. Furthermore, lack of exercise and over eating may also cause osteoarthritis as obesity causes extra load on the joint. Therefore, osteoarthritis is not specific to the elderly as it can affect people of all ages. When arthritis occurs serious, joint pains will cause patients to become lame and affect their health due to reduced activities, and may even cause fractures because of insufficient bone strength.

In addition to physical or medical therapy, knee arthroplasty can be applied to treat arthritis for chronic patients. knee arthroplasty are known as one of the three most important inventions in modern medicine. With the evolvement of medical manufacturing technology, arthritis patients can move freely and significantly improve the quality of life after receiving the operation. In the field of arthroplasty, it has the most mature technology and effect. Moreover, the life time of the knee arthroplasty is the most consistent. The survivorship of a general joint replacement is about 15 years, as the average human life expectancy increased, the situation that the knee arthroplasty needs to be revised due to prolonged survival life time also increased.

There is a columnar projection on the tibia component of a traditional knee arthroplastyarthroplasty for connecting with a tibia. In the surgery, there is a groove on the tibia so that the columnar projection can be inserted into the tibia along an axial direction. As the columnar projection is inserted into the tibia along a single direction(axial), it needs to be cemented between the columnar projection and the groove to allow the tibia to rotate freely within the knee arthroplastycomponent. In other words, the bone cement is used to enhance the bonding force between the tibia and the knee arthroplastytibial tray. Because the tibia component of the prior art is axially inserted into the tibia, the bone cement has better bearing capacity for normal stress when the knee bends, but the bearing capacity for shear stress is not enough. In order to resist shear stress, the use of bone cement must be increased to provide sufficient lateral bonding force. However, during the knee arthroplasty, excessive bone cement will make the implant hard to be removed and increases the difficulty of the revision surgery. Meanwhile, the process of bone cement removal will cause damage to the patient's bone.

### SUMMARY OF THE INVENTION

Therefore, one objective of the present invention is to provide a tibia component to solve the problem in the present technique. In addition to providing sufficient lateral bonding force, the tibia component of the present invention can also reduce the use of bone cement so as to facilitate the process of revision surgery.

According to an embodiment, the tibia component of the present invention is used for connecting with a tibia. The tibia component comprises a first tibia connecting structure and a second tibia connecting structure. The first tibia connecting structure can be radially inserted into a radial groove on the tibia, and the second tibia connecting structure can be axially inserted into a axial groove on the tibia.

In this embodiment, the tibia component provides radial and axial bonding force by radially inserting the first tibia connecting structure into the tibia and axially inserting the second tibia connecting structure into the tibia. With this method, the tibia component can be firmly connected with the tibia without utilizing a large amount of bone cement. That is to say, the tibia component of the present invention is helpful to the knee joint revision surgery.

Another objective of the present invention is to provide an knee arthroplasty to solve the problem in the current knee joint replacement.

According to an embodiment, the knee arthroplasty of the present invention comprises a tibia component used for connecting with a tibia. The tibia component comprises a first tibia connecting structure and a second tibia connecting structure. The first tibia connecting structure can be radially inserted into a radial groove on the tibia, and the second tibia connecting structure can be axially inserted into a axial groove on the tibia.

In this embodiment, the tibia component of the knee arthroplasty provides radial and axial bonding force by radially inserting the first tibia connecting structure into the tibia and axially inserting the second tibia connecting structure into the tibia. With this method, the tibia component of the knee arthroplasty can be firmly connected with a tibia without utilizing a large amount of bone cement. Consequently, the new arthroplasty design of the present invention is helpful for the total knee revision surgery.

Many other advantages and features of the present invention will be further understood by the following detailed description and the appended drawings.

### BRIEF DESCRIPTION OF THE DRAWINGS

FIG. 1 is a schematic diagram illustrating a knee arthroplasty according to an embodiment of the invention.

FIG. 2A is an expanded schematic diagram illustrating a tibia component of the knee arthroplasty in the FIG. 1.

FIG. 2B is a schematic diagram illustrating the combined tibia component in FIG. 2A.

FIG. 3A is a schematic diagram illustrating how the first assembly in FIG. 2A connects with a tibia.

FIG. 3B is a schematic diagram illustrating how the second assembly in FIG. 2A connects with a tibia.

To facilitate understanding, identical reference numerals have been used, where it is possible to designate identical elements that are common to the figures.

### DETAILED DESCRIPTION

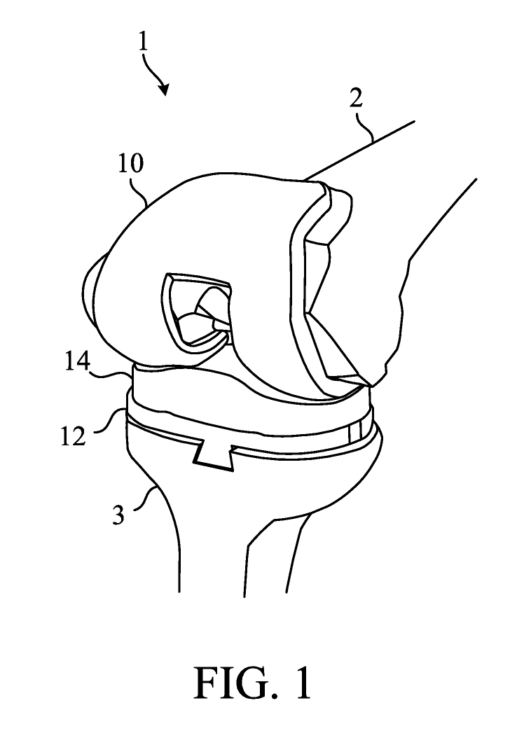

Please refer to FIG. 1. FIG. 1 is a schematic diagram illustrating an knee arthroplasty 1 according to an embodiment of the invention. The knee arthroplasty 1 can be implanted into a patient's leg, between a femur 2 and a tibia 3, to allow the femur 2 and the tibia 3 relative rotation and to support people's normal activities. As shown in FIG. 1, the knee arthroplasty 1 comprises a femur component 10, a tibia component 12 and a pad 14 between the femur component 10 and the tibia component 12. The femur component 10 and the tibia component 12 are pivoted with each other, while the pad 14 plays the role of reducing the friction between the femur component 10 and the tibia component 12. With the pad 14, the femur component 10 and the tibia component 12 can have smoother relative rotation and avoid excessive wear.

In this embodiment, the femur component 10 can connect with the patient's femur 2 and the tibia component 12 can connect with the patient's tibia 3. Therefore, with the relative rotation between the femur component 10 and the tibia component 12, the femur 2 and the tibia 3 can still work to support people's normal activities such as walking, running, jumping and squatting.

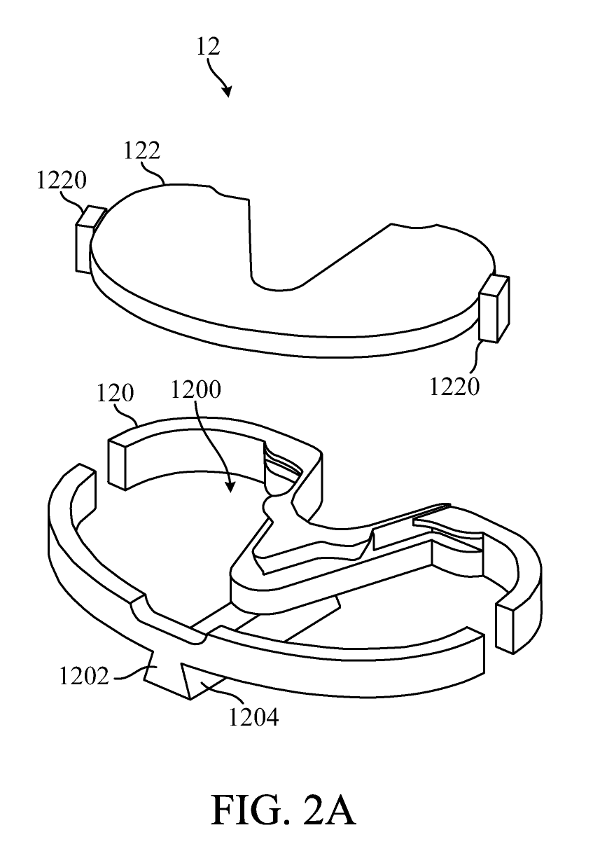

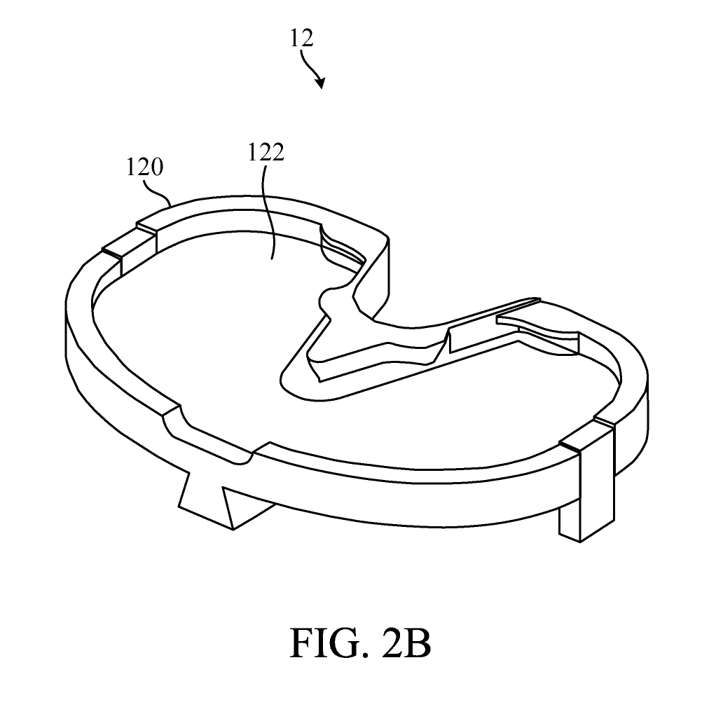

Please refer to FIG. 2A and FIG. 2B. FIG. 2A is an expanded schematic diagram illustrating a tibia component of the knee arthroplasty in the FIG. 1, and FIG. 2B is a schematic diagram illustrating the combined tibia component in FIG. 2A. As shown in FIG. 2A, the tibia component 12 comprises a first assembly 120 and a second assembly 122. The first assembly 120 have a receiving space 1200 to accommodate the second assembly 122. The second assembly 122 accommodated in the receiving space 1200 of the first assembly 120 forms the combined tibia component 12 (as shown in FIG. 2B). Furthermore, the tibia component 12 can hold the pad 14 and be pivoted with the femur component 10.

In FIG. 2A, a first tibia connecting structure 1202 is formed on the first assembly 120, and two second tibia connecting structures 1220 are formed on the second assembly 122. In this embodiment, the first tibia connecting structure 1202 can be a long wedge-shaped structure, and the width of the long wedge-shaped structure is gradually widened outward from the first assembly 120. The number of the second tibia connecting structure 1220 in this embodiment is two, but in practice may be one or more, in other words, the number of the second tibia connecting structure 1220 is not limited in the present invention. Furthermore, the first assembly 120 has openings corresponding to the second tibia connecting structure 1220 of the second assembly 122. When the second assembly 122 is accommodated in the receiving space 1200, the second tibia connecting structure 1220 can be accommodated in the opening and project from the first assembly 120.

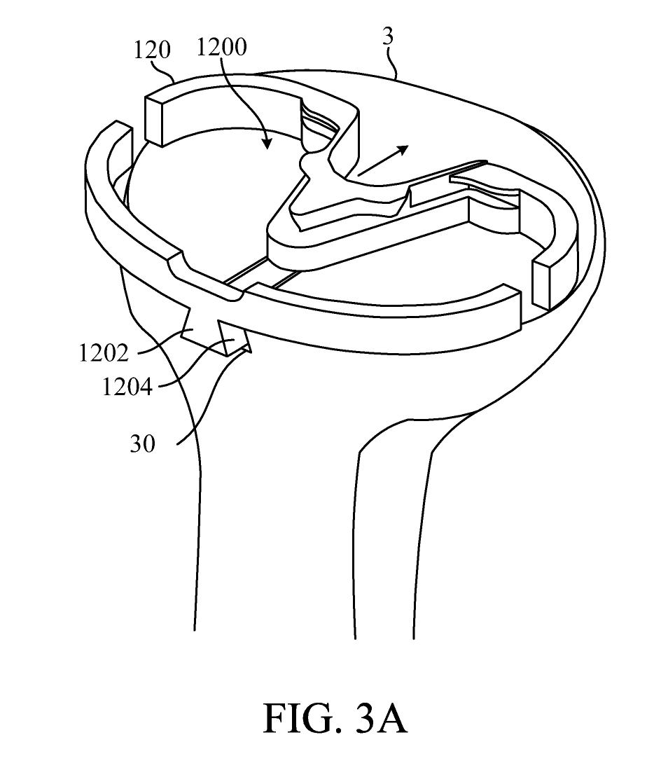

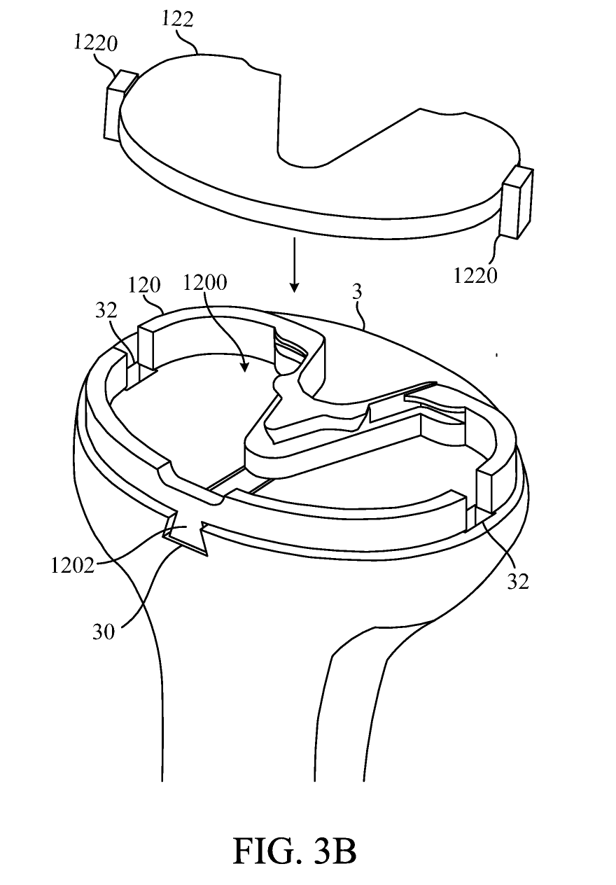

Please refer to FIG. 3A and FIG. 3B. FIG. 3A is a schematic diagram illustrating how the first assembly in FIG. 2A connects with a tibia, and FIG. 3B is a schematic diagram illustrating how the second assembly in FIG. 2A connects with a tibia. As shown in FIG. 3A, there is a radial groove 30 on the top of the tibia 3 (near the knee joint), and the section of the radial groove 30 corresponds to the section of the first tibia connecting structure 1202. In this embodiment, the radial groove 30 can be a long groove with a wedge-shaped section since the first tibia connecting structure 1202 is a long wedge-shaped structure.

When the tibia component 12 is being connected with the tibia 3 in a surgery, the first tibia connecting structure 1202 of the first assembly 120 can be aimed at and inserted into the radial groove 30 along a radial direction, as shown in FIG. 3. Please refer to FIG. 2A again. According to the figure, the first tibia connecting structure 1202 is a long wedge-shaped structure, with the width of the long wedge-shaped structure gradually widening outward from the first assembly 120, therefore, there are two inclines 1204 present on the first tibia connecting structure 1202. When the first tibia connecting structure 1202 is inserted into the radial groove 30, the two inclines 1204 can provide the tibia 3 normal and lateral supporting force, that is to say, the tibia component 12 can bear present normal stress and shear stress.

The above first tibia connecting structure 1202 uses the two inclines 1204 to support the tibia 3, however, in actual application, the first tibia connecting structure can be designed as another shape to achieve the same effect. For example, the first tibia connecting structure can be a long rectangular structure or a long circular structure. Thus, the principle of design of the first tibia connecting structure is for providing normal and lateral supporting force by radially inserting the first tibia connecting structure into the tibia, but, the section of the first tibia connecting structure is not limited in the present invention but depends on the demand of users.

In another embodiment, the surface of the first tibia connecting structure 1202 can be configured with a pad to limit the motion of the first assembly 120 in the axial direction, where the pad can be made in contact with the inner wall of the radial groove 30 when the first tibia connecting structure 1202 is inserted into the radial groove 30. In addition, the radial groove 30 can also be filled with bone cement to fix the first tibia connecting structure 1202. However, in this embodiment, the bone cement is only for fixing the first tibia connecting structure 1202, and the normal stress and shear stress from patient's activities are borne by the first tibia connecting structure 1202, therefore, the use of bone cement can be greatly reduced.

After the first assembly 120 is combined with the tibia 3 by inserting the first tibia connecting structure 1202 into the radial groove 30, the second assembly 122 can be axially inserted into the receiving space 1200 of the first assembly 120. As shown in FIG. 3B, there is another axial groove 32 on the top of the tibia 3, and the position of the axial groove 32 corresponds to the second tibia connecting structure 1220 of the second assembly 122. After the second assembly 122 is accommodated in the receiving space 1200 of the first assembly 120, the second tibia connecting structure 1220 can be axially inserted into the axial groove 32 to limit the motion of the first assembly 120 and the tibia component 12 in the radial direction. The axial groove 32 can also be filled with bone cement to fix the second tibia connecting structure 1220. Likewise, the normal stress and shear stress from patient's activities are mainly borne by the first tibia connecting structure 1202, thus, the use of bone cement can be greatly reduced.

In this embodiment, the second assembly 122 is but not limited to a long square pillar, in actual application, the second assembly can be a long column or a long pillar with other section shapes. In addition, the principle of design of the second tibia connecting structure 1220 is fixing and limiting the motion of the tibia component 12 in the radial direction, therefore, the length of the projection of the second tibia connecting structure 1220 and depth of the axial groove 32 are not limited in the present invention but depend on the demand of users.

The above tibia component comes of the combination of the first assembly and the second assembly; however, the tibia component can be designed as an integral of both assemblies. According to another embodiment, the tibia component can be designed as a body with a movable first tibia connecting structure and a movable second tibia connecting structure, that is to say, the tibia component is not disassembled into the first assembly and the second assembly, but designed as an integral including the first tibia connecting structure and the second tibia connecting structure. In this embodiment, the first tibia connecting structure and the second tibia connecting structure may both be designed as a movable part, or just one of them. The following are descriptions about the method of fixing a variety of integral tibia components with a tibia.

In one embodiment of the present invention, the first tibia connecting structure of the above integral tibia component is fixed but the second tibia connecting structure is movable. The installation of the tibia component consists of adjusting the second tibia connecting structure to a standby position, and then inserting the first tibia connecting structure into the radial groove. For the shape of the first tibia connecting structure and the radial groove, please refer to FIG. 3A and FIG. 3B. The motion of the tibia component in the axial direction is limited when the first tibia connecting structure is inserted into the radial groove. Then, inserting the second tibia connecting structure into the axial groove to limit the motion of the tibia component in the radial direction. After the above process, the tibia component is firmly connected with the tibia.

In another embodiment, the first tibia connecting structure of the above integral tibia component is movable but the second tibia connecting structure is fixed. The installation of the tibia component consists of inserting the second tibia connecting structure into the axial groove, and then inserting the first tibia connecting structure into the radial groove. Moreover, if the first tibia connecting structure and the second tibia connecting structure are both movable, the installation of the tibia component consists of putting the tibia component on a standby position, then, inserting the first tibia connecting structure and the second tibia connecting structure into the radial groove and the axial groove, respectively. In addition, the above integral tibia components use the first tibia connecting structure and the second tibia connecting structure to provide the radial and axial bonding force so that the use of bone cement can be greatly reduced.

According to the above, the tibia component of the knee arthroplasty of the present invention comprises two assemblies. The first assembly comprises a first tibia connecting structure to be radially inserted into a radial groove of a tibia. The second assembly can be accommodated in the first assembly and comprises a second tibia connecting structure to be axially inserted into a axial groove of a tibia. The first tibia connecting structure and the second tibia connecting structure provide the radial and axial bonding force that the tibia component needs to bear the normal stress and shear stress from patient's activities. Accordingly, compared to the prior art, the tibia component of the knee arthroplasty of the present invention can be firmly connected with a tibia without utilizing a large amount of bone cement, which is helpful for knee joint revision.

With the examples and explanations mentioned above, the features and spirits of the invention are hopefully well described. Importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

1. A tibia component, used in a knee arthroplasty for connecting with a tibia, comprising:
at least one first tibia connecting structure, inserted into a radial groove on the tibia along a radial direction to limit the motion of the tibia component in the axial direction; and
at least one second tibia connecting structure, inserted into an axial groove on the tibia along an axial direction to limit the motion of the tibia component in the radial direction.

2. The tibia component of claim 1, further comprising:
a first assembly with a receiving space, wherein the first tibia connecting structure is formed on the first assembly; and
a second assembly, accommodated in the receiving space, wherein the second tibia connecting structure is formed on the second assembly.

3. The tibia component of claim 1, wherein the first tibia connecting structure is a long wedge-shaped structure, and the width of the long wedge-shaped structure is gradually widened outward from the tibia component.

4. The tibia component of claim 3, further comprising a pad configured on the surface of the long wedge-shaped structure, wherein the pad can be made in contact with the inner wall of the radial groove when the long wedge-shaped structure is inserted into the radial groove.

5. The tibia component of claim 1, wherein the radial groove is filled with bone cement to connect with the first tibia connecting structure, the axial groove is filled with bone cement to connect with the second tibia connecting structure.

6. A knee arthroplasty, used to connect a tibia with a femur, comprising:
a femur component, used to connect with the femur; and
a tibia component, wherein the tibia component and the femur component are pivoted with each other so that the tibia component can be relatively rotated to the femur component, and the tibia component is used for connecting with the tibia, comprising:
at least one first tibia connecting structure, inserted into a radial groove on the tibia along a radial direction to limit the motion of the tibia component in the axial direction; and
at least one second tibia connecting structure, inserted into a axial groove on the tibia along a axial direction to limit the motion of the tibia component in the radial direction.

7. The knee arthroplasty of claim 6, wherein the tibia component further comprises:
a first assembly with a receiving space, wherein the first tibia connecting structure is formed on the first assembly; and
a second assembly, accommodated in the receiving space, wherein the second tibia connecting structure is formed on the second assembly.

8. The knee arthroplasty of claim 6, wherein the first tibia connecting structure is a long wedge-shaped structure, and the width of the long wedge-shaped structure is gradually widened outward from the tibia component.

9. The knee arthroplasty of claim 8, further comprising a pad configured on the surface of the long wedge-shaped structure, wherein the pad can be made in contact with the inner wall of the radial groove when the long wedge-shaped structure is inserted into the radial groove.

10. The knee arthroplasty of claim 6, wherein the radial groove is filled with bone cement to connect with the first tibia connecting structure, while the axial groove is filled with bone cement to connect with the second tibia connecting structure.
